(19) United States
(12) Patent Application Publication
(10) Pub. No.: US 2013/0158040 A1
(43) Pub. Date: Jun. 20, 2013

(54) DIAZAINDOLE INHIBITORS OF CRAC

(71) Applicant: Hoffmann-La Roche Inc., Nutley, NJ (US)

(72) Inventors: Muzaffar Alam, Clifton, NJ (US); Niala Bhagirath, Bloomfield, NJ (US); Daisy Joe Du Bois, Menlo Park, CA (US); Ronald Charles Hawley, Glen Ridge, NJ (US); Ana Elena Minatti, Santa Monica, CA (US); Joshua Kennedy-Smith, New York, NY (US); Robert Stephen Wilhelm, Los Altos, CA (US)

(73) Assignee: Hoffmann-La Roche Inc., Nutley, NJ (US)

(21) Appl. No.: 13/716,548

(22) Filed: Dec. 17, 2012

Related U.S. Application Data
(60) Provisional application No. 61/577,854, filed on Dec. 20, 2011.

(51) Int. Cl. C07D 487/04

(52) U.S. Cl. CPC C07D 487/04
USPC 514/249; 544/280; 514/265.1; 544/350

(57) ABSTRACT

Disclosed are compounds of Formula (I):

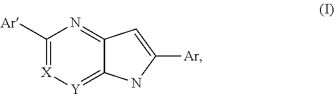

useful for treatment of autoimmune and inflammatory diseases associated with IL-2 inhibition via modulation of calcium release-activated calcium (CRAC) channels. Also disclosed are methods of making and using the compounds for treatment of diseases associated with CRAC channels.

### PRIORITY TO RELATED APPLICATION(S)

This application claims the benefit of U.S. Provisional Application No. 61/577,854, filed Dec. 20, 2011, which is hereby incorporated by reference in its entirety.

### RELATED APPLICATION(S)

This application is related to U.S. application Ser. No. 12/888,701, filed on Sep. 23, 2010, the entire contents of which are incorporated by reference herein.

### FIELD OF THE INVENTION

This invention pertains to compounds useful for treatment of autoimmune and inflammatory diseases associated with IL-2 inhibition via modulation of calcium release-activated calcium channels.

### BACKGROUND OF THE INVENTION

The cytokine interleukin 2 (IL-2) is a T-cell mitogen important for T-cell proliferation and as a B cell growth factor. Because of its effects on T cells and B cells, IL-2 is recognized as an important regulator of immune responses. IL-2 is involved in inflammation, tumor progression and hematopoiesis, and IL-2 affects the production of other cytokines such as TNA alpha, TNF beta, IFN gamma. Inhibition of IL-2 production thus is relevant to immunosuppression therapies and treatment of inflammatory and immune disorders.

T-cell antigen binding in inflammatory events leads to T-cell initiated calcium influx by calcium release-activated calcium channels (CRAC). IL-2 secretion by T-cells occurs in response to calcium ion influx. Modulation of CRAC thus provides a mechanism for control of production of IL-2 and other cytokines associated with inflammation. CRAC inhibition has been recognized as a potential route to therapies for rheumatoid arthritis, asthma, allergic reactions and other inflammatory conditions (see, e.g., Chang et al., Acta Pharmacologica Sinica (2006) Vol. 7, 813-820), and CRAC inhibitors have been shown to prevent antigen-induced airway eosinophilia and late phase asthmatic responses via Th2 cytokine inhibition in animal models (Yoshino et al., Eur. J. Pharm. (2007) Vol. 560(2), 225-233). There is, accordingly, a need for CRAC inhibitors.

### SUMMARY OF THE INVENTION

The invention provides a compound of Formula (I):

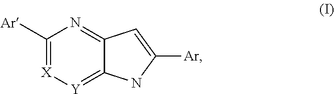

wherein:
one of X or Y is C and the other is N;
Ar is unsubstituted cycloalkyl, unsubstituted phenyl or phenyl mono- or bi-substituted independently with halogen;
Ar′ is—phenyl, unsubstituted or mono- or bi-substituted independently with —SO₂N(CH₃)₂, lower alkyl or —C(O)OCH₃, or
heteroaryl, unsubstituted or mono- or bi-substituted independently with lower alkyl or haloalkyl;
   or a pharmaceutically acceptable salt thereof.

The invention also provides for pharmaceutical compositions comprising the compounds, methods of using the compounds, and methods of preparing the compounds.

All documents cited to or relied upon below are expressly incorporated herein by reference.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like.

“Alkoxy” and “alkyloxy”, which may be used interchangeably, mean a moiety of the formula —OR, wherein R is an alkyl moiety as defined herein. Examples of alkoxy moieties include, but are not limited to, methoxy, ethoxy, isopropoxy, and the like.

“Aryl” means a monovalent cyclic aromatic hydrocarbon moiety having a mono-, bi- or tricyclic aromatic ring. The aryl group can be optionally substituted as defined herein. Examples of aryl moieties include, but are not limited to, phenyl, naphthyl, phenanthryl, fluorenyl, indenyl, pentalenyl, azulenyl, oxydiphenyl, biphenyl, methylenediphenyl, aminodiphenyl, diphenylsulfidyl, diphenylsulfonyl, diphenylisopropylidenyl, benzodioxanyl, benzofuranyl, benzodioxylyl, benzopyranyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like, including partially hydrogenated derivatives thereof, each being optionally substituted.

“Cycloalkyl” means a monovalent saturated carbocyclic moiety having mono- or bicyclic rings. Preferred cycloalkyl are unsubstituted or substituted with alkyl. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated (cycloalkenyl) derivatives thereof.

“Heteroaryl” means a monocyclic or bicyclic radical of 5 to 12 ring atoms having at least one aromatic ring containing one, two, three or four ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, thienyl, benzothienyl, thiophenyl, furanyl, pyranyl, pyridyl, pyrrolyl, pyrazolyl, pyrimidyl, quinolinyl, isoquinolinyl, benzofuryl, benzothiophenyl, benzothiopyranyl, benzimidazolyl, benzooxazolyl, benzooxadiazolyl, benzothiazolyl, benzothiadiazolyl, benzopyranyl, indolyl, isoindolyl, tetrazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like, including partially hydrogenated derivatives thereof, each optionally substituted.

The terms “halo”, “halogen” and “halide”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or
salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalian class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Arthritis” means diseases or conditions damage to joints of the body and pain associated with such joint damage. Arthritis includes rheumatoid arthritis, osteoarthritis, psoriatic arthritis, septic arthritis and gouty arthritis.

“Pain” includes, without limitation, inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes: preventing the disease state, i.e. causing the clinical symptoms of the disease state not to develop in a subject that may be exposed to or predisposed to the disease state, but does not yet experience or display symptoms of the disease state:

inhibiting the disease state, i.e., arresting the development of the disease state or its clinical symptoms, or
relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen sulfur or nitrogen atom in the structures herein indicates the presence of a hydrogen atom unless indicated otherwise. Where a nitrogen-containing heteroaryl ring is shown with an open valency on a nitrogen atom, and variables such as R^{a}, R^{b} or R^{c} are shown on the heteroaryl ring, such variables may be bound or joined to the open valency nitrogen. Where a chiral center exists in a structure but no specific stereochemistry is shown for the chiral center, both enantiomers associated with the chiral center are encompassed by the structure. Where a structure shown herein may exist in multiple tautomeric forms, all such tautomers are encompassed by the structure. The atoms represented in the structures herein are intended to encompass all naturally occurring isotopes of such atoms. Thus, for example, the hydrogen atoms represented herein are meant to include deuterium and tritium, and the carbon atoms are meant to include C¹³ and C¹⁴ isotopes.

In one embodiment, the invention provides for a compound of Formula (I):

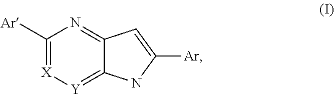

wherein:
one of X or Y is C and the other is N;
Ar is unsubstituted cycloalkyl, unsubstituted phenyl or phenyl mono- or bi-substituted independently with halogen;
Ar′ is—phenyl, unsubstituted or mono- or bi-substituted independently with —SO₂N(CH₃)₂, lower alkyl or —C(O)OCH₃, or
heteroaryl, unsubstituted or mono- or bi-substituted independently with lower alkyl or haloalkyl;
   or a pharmaceutically acceptable salt thereof.

In another embodiment, the invention provides for a compound of Formula (I) wherein X is C and Y is N.

In another embodiment, the invention provides for a compound of Formula (I) wherein X is N and Y is C.

In another embodiment, the invention provides for a compound of Formula (I) wherein Ar is phenyl mono- or bi-substituted independently with halogen, and Ar′ is phenyl, unsubstituted or mono- or bi-substituted independently with —SO₂N(CH₃)₂, lower alkyl or —C(O)OCH₃.

In another embodiment, the invention provides for a compound of Formula (I) wherein Ar is phenyl mono- or bi-substituted independently with halogen, and Ar is heteroaryl, unsubstituted or mono- or bi-substituted independently with lower alkyl or haloalkyl.

In another embodiment, the invention provides for a compound of Formula (I) wherein Ar cyclohexyl and Ar′ is phenyl, unsubstituted or mono- or bi-substituted independently with —SO₂N(CH₃)₂, lower alkyl or —C(O)OCH₃.

In another embodiment, the invention provides for a compound of Formula (I) wherein Ar is cyclohexyl and Ar′ is heteroaryl, unsubstituted or mono- or bi-substituted independently with lower alkyl or haloalkyl.

In another embodiment, the invention provides for a compound of Formula (I) wherein Ar is phenyl bisubstitued independently with chlorine or fluorine.

In another embodiment, the invention provides for a compound of Formula (I) wherein Ar is cyclohexyl.

In another embodiment, the invention provides for a compound of Formula (I) wherein Ar′ is N,N-trimethyl-benzenesulfonamide or methyl-benzoic acid methyl ester.

In another embodiment, the invention provides for a compound of Formula (I) wherein Ar′ is pyrazolyl, unsubstituted or mono- or bi-substituted independently with methyl or —CF₃.

In another embodiment, the invention provides for a compound of Formula (I) wherein the compound is:
4-[6-(2-chloro-6-fluoro-phenyl)-5H-pyrrolo[3,2-d]pyrimidin-2-yl]-3,N,N-trimethyl-benzenesulfonamide;
6-(2-Chloro-phenyl)-2-(2-methyl-5-trifluoromethyl-2H-pyrazol-3-yl)-5H-pyrrolo[2,3-b]pyrazine;
4-[6-(2-Chloro-phenyl)-5H-pyrrolo[2,3-b]pyrazin-2-yl]-3-methyl-benzoic acid methyl ester;
4-(6-(2-Chlorophenyl)-5H-pyrrolo[2,3-b]pyrazin-2-yl)-N,N,3-trimethylbenzenesulfonamide;
6-(2-chloro-6-fluorophenyl)-2-(1-methyl-3-(trifluoromethyl)-1H-pyrazol-5-yl)-5H-pyrrolo[2,3-b]pyrazine;
6-Cyclohexyl-2-(1-methyl-3-(trifluoromethyl)-1H-pyrazol-5-yl)-5H-pyrrolo[2,3-b]pyrazine; or
4-(6-cyclohexyl-5H-pyrrolo[2,3-b]pyrazin-2-yl)-N,N,3-trimethylbenzenesulfonamide.

In another embodiment, the invention provides for a pharmaceutical composition, comprising a therapeutically effective amount of a compound according to Formula (I) and a pharmaceutically acceptable carrier.

In another embodiment, the invention provides for a compound according to Formula (I) for use as a therapeutically active substance.

In another embodiment, the invention provides for a use of a compound according to Formula (I) for the treatment or prophylaxis of arthritis or a respiratory disorder.

In another embodiment, the invention provides for a use of a compound according to Formula (I) for the preparation of a medicament for the treatment or prophylaxis of arthritis or a respiratory disorder.

In another embodiment, the invention provides for a compound according to Formula (I) for the treatment or prophylaxis of arthritis or a respiratory disorder.

In another embodiment, the invention provides for a method for treating arthritis, comprising the step of administering a therapeutically effective amount of a compound according to Formula (I) to a subject in need thereof.

In another embodiment, the invention provides for a method for treating a respiratory disorder selected from chronic obstructive pulmonary disorder (COPD), asthma, and bronchospasm, comprising the step of administering a therapeutically effective amount of a compound according to Formula (I) to a subject in need thereof.

In a further embodiment, provided is an invention as hereinbefore described.

The invention also provides methods for treating a disease or condition mediated by or otherwise associated with a CRAC receptor, the method comprising administering to a subject in need thereof an effective amount of a compound of the invention.

The invention also provides methods for treating an inflammatory, respiratory or diabetes condition, the method comprising administering to a subject in need thereof an effective amount of a compound of the invention together with an effective amount of a CRAC inhibitor.

The disease may be an inflammatory disease such as arthritis, and more particularly rheumatoid arthritis, osteoarthritis, psoriasis, allergic dermatitis, asthma, chronic obstructive pulmonary disease, airways hyper-responsiveness, septic shock, glomerulonephritis, irritable bowel disease, and Crohn's disease.

The disease may be a pain condition, such as inflammatory pain; surgical pain; visceral pain; dental pain; premenstrual pain; central pain; pain due to burns; migraine or cluster headaches; nerve injury; neuritis; neuralgias; poisoning; ischemic injury; interstitial cystitis; cancer pain; viral, parasitic or bacterial infection; post-traumatic injury; or pain associated with irritable bowel syndrome.

The disease may be a respiratory disorder, such as chronic obstructive pulmonary disorder (COPD), asthma, or bronchospasm, or a gastrointestinal (GI) disorder such as Irritable Bowel Syndrome (IBS), Inflammatory Bowel Disease (IBD), biliary colic and other biliary disorders, renal colic, diarrhea-dominant IBS, pain associated with GI distension.

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

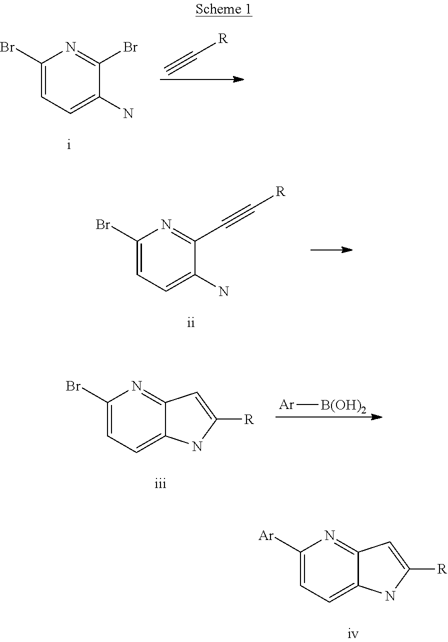

As shown in Scheme 1, halogen substituted heterocyclic amines of type i can be reacted under Sonogashira coupling conditions with an appropriate terminal alkyne to give the alkyne substituted heterocyclic amine ii, where R=aryl, heteroaryl, cycloalkyl, heterocycloalkyl, or alkyl. Conversion of alkynyl amine ii, in the presence of base or a transition metal catalyst, then gives 2-substituted-5-halo-4-azaindole of type iii. Suzuki coupling of indole iii with an appropriate boronic acid or ester then gives 2-substituted-5-aryl-4-azaindole iv.

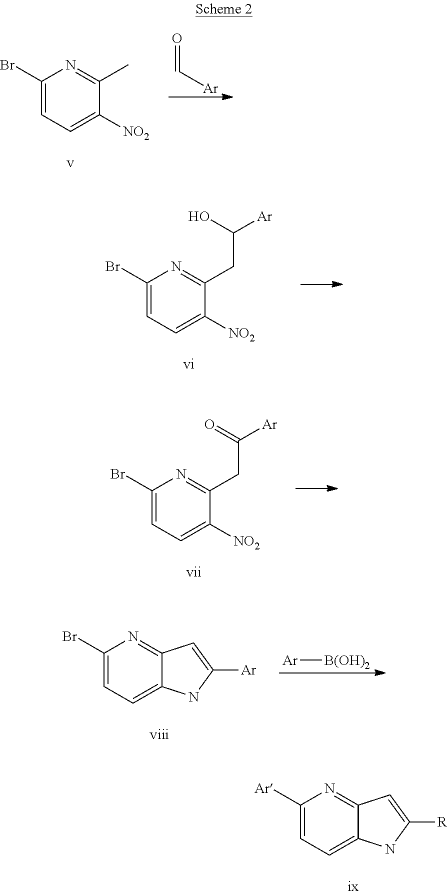

As shown in Scheme 2, 3-nitro-picoline v, can be converted to the nitropyridine substituted acetophenone vii via the intermediacy of alcohol vi. Dual reduction and cyclization then gives 4-aza-indole viii, which can be converted to 2,5-diaryl-4-azaindole ix by means of a Suzuki coupling with an appropriate boronic acid or ester.

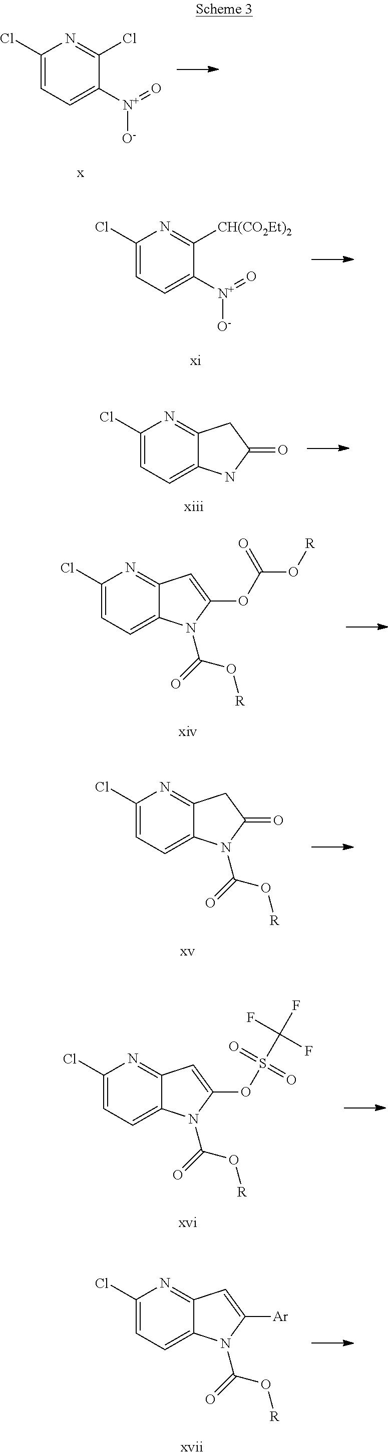

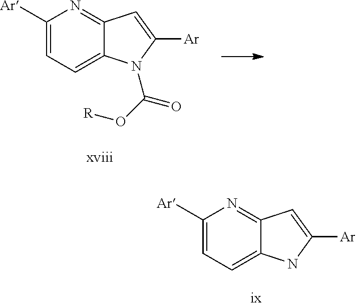

As shown in Scheme 3, 2,6-dichloro-3-nitro-pyridine v, can be transformed to 4-aza-oxindole xii in two steps via malonate xi. Conversion of oxindole xii to triflate xvi can be accomplished by addition and selective removal of an intermediate carbonate as reflected in structures xiv and xv. Sequential Suzuki couplings on triflate xvi with the appropriate boronic acids or esters then provides carbonate protected indole xviii. Compounds such as these can then be converted to 2,5-diaryl-4-azaindole ix under basic conditions.

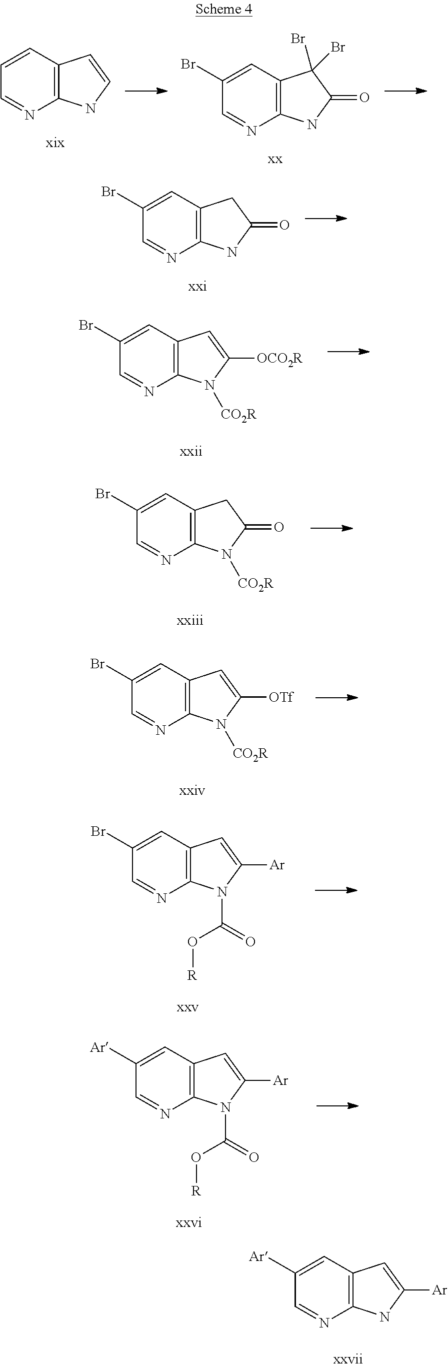

As shown in Scheme 4, 2,5-diaryl-7-azaindole xxvii can be produced in a manner similar to that shown in Scheme 3 substituting bromo oxindole xxi. This material can be prepared in two steps from 7-azaindole via the intermediacy tribromo oxindole xx.

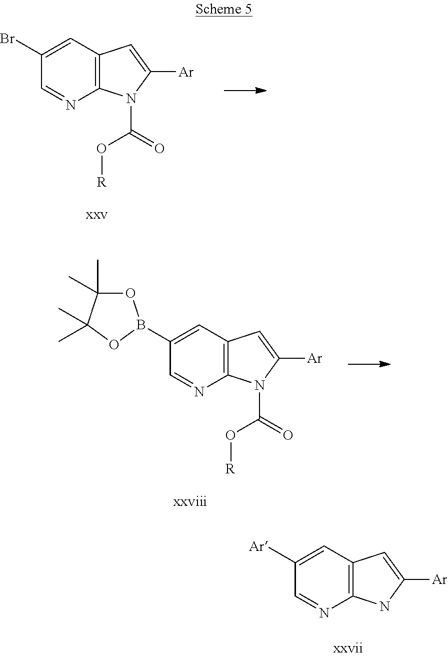

As shown in Scheme 5, carbonate protected 5-bromo-7-azaindole xxv from Scheme 4 can also be converted to boronic ester xxiii. Suzuki coupling with aryl halides or triflates then provides access to 2,5-diaryl-7-azaindole xxvii.

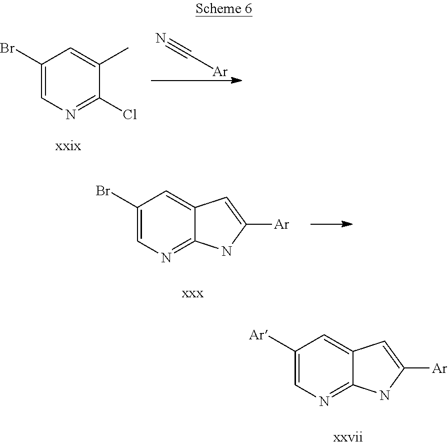

As shown in Scheme 6, 5-bromo-2-chloro-3-methylpyridine xxix can be reacted with an appropriate benzonitrile and base to provide 5-bromo-7-azaindole xxx. This indole xxx can then be converted to 2,5-diaryl-7-azaindole xxvii by means of a Suzuki coupling with an appropriate boronic acid or ester.

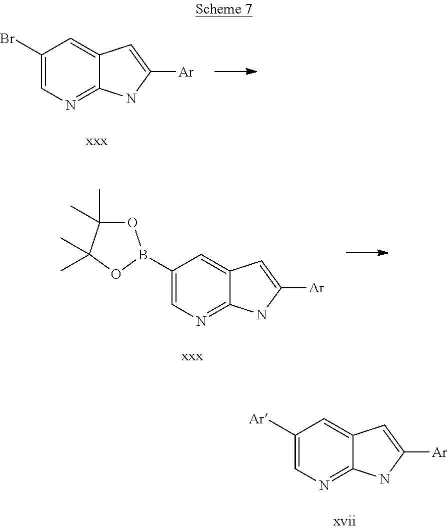

As shown in Scheme 7, 5-bromo-7-azaindole xxx from Scheme 6 can also be converted to boronic ester xxxi. Suzuki coupling with aryl halides or triflates then provides access to 2,5-diaryl-7-azaindole xxvii.

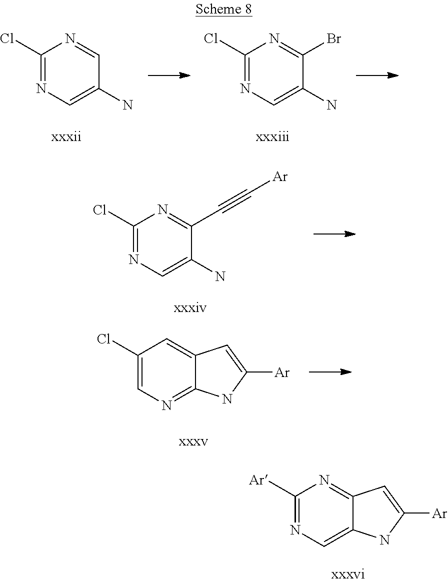

As shown in Scheme 8, pyrimidine xxxii can be brominated to xxxiii and transformed to 4,6-diazaindole xxxv using a Sonogashira/base-mediated cyclization strategy. Suzuki coupling with an appropriate boronic acid or ester then provides access to the 2,5-diaryl-4,6-diazaindole xxxv.

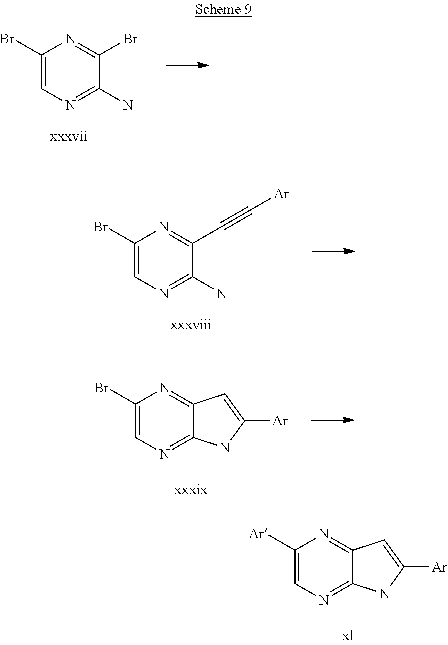

As shown in Scheme 9, 2-amino-3,5-dibromopyrazine can be transformed in a manner similar to that shown in Scheme 8 to provide 2,5-diaryl-4,7-diazaindole xl.

Many variations on the procedure of the above Schemes are possible and will suggest themselves to those skilled in the art. Specific details for producing compounds of the invention are described in the Examples section below.

### Utility

The compounds of the invention are usable for the treatment of a wide range of inflammatory diseases and conditions such as arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions. The subject compounds would be useful for the treatment of pulmonary disorders or lung inflammation, including adult respiratory distress syndrome, pulmonary sarcoidosis, asthma, silicosis, and chronic pulmonary inflammatory disease.

Further, compounds of the invention are useful for treating respiratory disorders, including chronic obstructive pulmonary disorder (COPD), asthma, bronchospasm, and the like.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

### EXAMPLES

The following preparations and examples are given to enable those skilled in the art to more clearly understand and to practice the present invention. They should not be considered as limiting the scope of the invention, but merely as being illustrative and representative thereof.

Unless otherwise stated, all temperatures including melting points (i.e., MP) are in degrees celsius (° C.). It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Part I: Preparation of Certain Intermediates

### Intermediate 1

### Trifluoro-methanesulfonic acid 2-ethyl-5-pyridin-3-yl-2H-pyrazol-3-yl ester

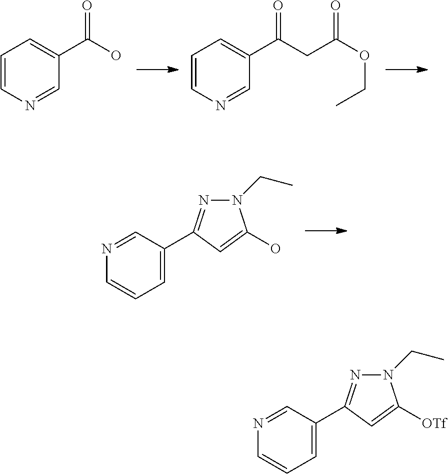 [SMILES: O=C(O)c1cccnc1 ; CCOC(=O)CC(=O)c1cccnc1 ; CCn1nc(-c2cccnc2)cc1O ; CCn1nc(-c2cccnc2)cc1OS(=O)(=O)C(F)(F)F]

3-Oxo-3-pyridin-3-yl-propionic acid ethyl ester: To nicotinic acid (20 g, 162.6 mmol) dissolved in dry THF was added CDI (30.95 g, 273.9 mmol) at 10° C. The mixture was stirred at RT for 1 h. In another flask the potassium salt of diethyl malonate (40.17 g, 245.1 mmol) and MgCl₂ (18.05 g, 189.59 mmol) were suspended in THF and heated to 50° C. for 4 h. The nicotinic acid/CDI mixture was then added to it and the entire mixture stirred at RT for 16 h. After completion, the mixture was quenched with water and extracted with EtOAc. The organic phase was washed with brine, dried over Na₂SO₄ and concentrated. The crude compound was purified by column chromatography using 30% EtOAc-Hexane as an eluent to give 3-oxo-3-pyridin-3-yl-propionic acid ethyl ester (7.8 g, 24.7%).

2-Ethyl-5-pyridin-3-yl-2H-pyrazol-3-ol: To 3-oxo-3-pyridin-3-yl-propionic acid ethyl ester (500 mg, 3.57 mmol) in AcOH was added ethylhydrazine oxalate (231.9 mg, 3.86 mmol) and the mixture refluxed for 16 h. After which, the AcOH was evaporated and crude mass neutralized with aq. Na₂CO₃ solution. Following extraction with EtOAc, the organic phase was washed with brine, dried over Na₂SO₄ and concentrated. The crude material was purified by column chromatography using 2% MeOH-DCM as an eluent to give 2-ethyl-5-pyridin-3-yl-2H-pyrazol-3-ol (110 mg, 22.5%) as a yellow solid.

Trifluoro-methanesulfonic acid 2-ethyl-5-pyridin-3-yl-2H-pyrazol-3-yl ester: To a solution of 2-ethyl-5-pyridin-3-yl-2,4-dihydro-pyrazol-3-one (200 mg, 1.058 mmol) in THF, cooled to 0° C., was added NaH (33 mg, 1.37 mmol) followed by N,N-bis(Trifluoromethanesulfonyl)aniline (567 mg, 1.58 mmol). The resulting mixture was stirred at 25° C. for 1 h, after which, it was quenched with ice-water and extracted with EtOAc. The organic phase was washed with 1 N NaOH, dried over Na₂SO₄ and concentrated. The crude material was then purified by column chromatography using 20% EtOAc-Hexane as an eluent to give trifluoro-methanesulfonic acid 2-ethyl-5-pyridin-3-yl-2H-pyrazol-3-yl ester (170 mg, 50%).

### Intermediate 2

### Trifluoro-methanesulfonic acid 5-methyl-2-pyridin-3-yl-thiazol-4-yl ester

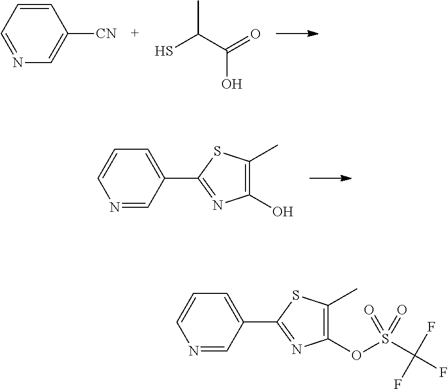

5-Methyl-2-pyridin-3-yl-thiazol-4-ol: To nicotinonitrile (2 g, 19.21 mmol) and 2-mercapto-propionic acid (2.04 g, 19.21 mmol) was added pyridine (0.38 ml, 4.80 mmol). The mixture heated to 100° C. After 3 h the mixture was cooled to rt, diluted with EtOH (20 ml) and stirred for 10 min. The resulting solid was filtered, washed with ether and dried under vacuum to give 5-methyl-2-pyridin-3-yl-thiazol-4-ol (2.5 g, 67.7%).

Trifluoro-methanesulfonic acid 5-methyl-2-pyridin-3-yl-thiazol-4-yl ester: To a solution of 5-methyl-2-pyridin-3-yl-thiazol-4-ol (300 mg, 1.56 mmol) in THF, cooled to 0° C., was added NaH (24 mg, 48.70 mmol) followed by N,N-Bis(Trifluoromethanesulfonyl)aniline (357 mg, 1.81 mmol). The mixture was stirred at 25° C. for 1 h, after which it was quenched with ice-water and extracted with EtOAc. The organic phase was washed with 1 N NaOH, dried over Na₂SO₄ and concentrated. The crude compound was purified by column chromatography using 20% EtOAc-Hexane as an eluent to obtain trifluoro-methanesulfonic acid 5-methyl-2-pyridin-3-yl-thiazol-4-yl ester (200 mg, 40%).

### Intermediate 3

### Trifluoro-methanesulfonic acid 2-methyl-5-trifluoromethyl-2H-pyrazol-3-yl ester

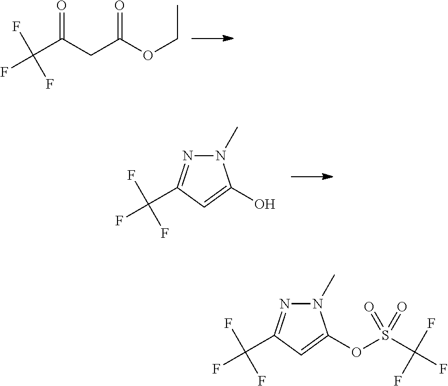 [SMILES: CCOC(=O)CC(=O)C(F)(F)F ; Cn1nc(C(F)(F)F)cc1O ; Cn1nc(C(F)(F)F)cc1OS(=O)(=O)C(F)(F)F]

2-Methyl-5-trifluoromethyl-2H-pyrazol-3-ol: To a solution of 4,4,4-Trifluoro-3-oxo-butyric acid ethyl ester (10 g, 54.34 mmol) in EtOH (40 ml) was added methyl hydrazine (2.9 ml, 54.34 mmol) and HCl (2 ml). The mixture was refluxed for 2 days, after which point the EtOH was evaporated and water was added to the reaction mixture. This was then extracted with EtOAc and the organic phase was evaporated to obtain 2-Methyl-5-trifluoromethyl-2H-pyrazol-3-ol (8 g, 89%) as an off-white solid.

Trifluoro-methanesulfonic acid 2-methyl-5-trifluoromethyl-2H-pyrazol-3-yl ester: To a solution of 2-Methyl-5-trifluoromethyl-2H-pyrazol-3-ol (5 g, 30.1 mmol) in DCM (80 mL) at 0° C. was added TEA (8.42 mL, 60.2 mmol), followed by drop wise addition of Tf₂O (7.47 mL, 45.1 mmol). The reaction mixture was allowed to warm to 25° C. and stirred for 1 h. Water was then added to quench the reaction and it was extracted with DCM. The organic phase was then washed with brine, dried over Na₂SO₄, and concentrated in vacuo to give Trifluoro-methanesulfonic acid 2-methyl-5-trifluoromethyl-2H-pyrazol-3-yl ester (5.5 g, 80%) which was sufficiently pure for use in further reactions.

### Intermediate 4

### Trifluoro-methanesulfonic acid 2-ethyl-5-trifluoromethyl-2H-pyrazol-3-yl ester

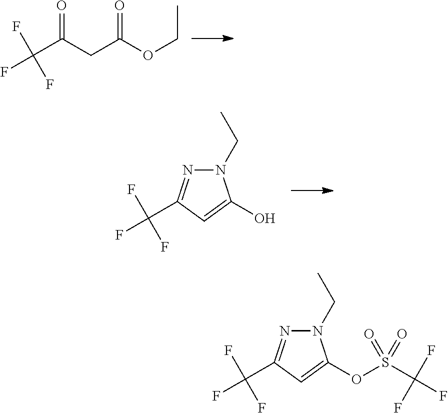 [SMILES: CCOC(=O)CC(=O)C(F)(F)F ; CCn1nc(C(F)(F)F)cc1O ; CCn1nc(C(F)(F)F)cc1OS(=O)(=O)C(F)(F)F]

Intermediate 3 can be prepared in a manner identical to that used for Intermediate 2 substituting ethyl hydrazine oxalate in the condensation step. An alternate procedure is also described here:

ethyl-3-(trifluoromethyl)-1H-pyrazol-5(4H)-one: A mixture of ethyl 4,4,4-trifluoroacetoacetate (11.0 g, 59.7 mmol) and ethyl hydrazine oxalate (8.96 g, 59.7 mmol) in acetic acid (60 ml) was heated at 120° C. in a microwave reactor for 1.5 h. After irradiation the reaction mixture was poured into ice water, extracted with EtOAc. The organic phase was then washed with brine, dried over Na₂SO₄, filtered, concentrated under reduced pressure, and the crude material purified by flash chromatography (5-10% EtOAc/hexanes) to give 2-Ethyl-5-trifluoromethyl-2H-pyrazol-3-ol (4.62 g, 43%) as a yellow solid.
ethyl-3-(trifluoromethyl)-1H-pyrazol-5-yl trifluoromethanesulfonate: To a solution of 2-Ethyl-5-trifluoromethyl-2H-pyrazol-3-ol (4.41 g, 24.5 mmol) in CH₂CH₂ (100 ml) and DIPEA (4.75 g, 36.7 mmol) at 0° C. was added trifluoromethane sulfonic anhydride (8.98 g, 31.8 mmol) dropwise. The mixture was stirred at 0° C. for 1 hour, then a cold solution of aqueous ammonium chloride and dichloromethane was added. The mixture was partitioned, and the organic phase washed with brine, dried over Na₂SO₄, filtered, concentrated under reduced pressure, and the crude material purified by filtering through a pad of silica (8% EtOAc/Hexanes) to give 1-ethyl-3-(trifluoromethyl)-1H-pyrazol-5-yl trifluoromethanesulfonate (6.12 g, 80%) as a yellow oil.

### Intermediate 5

### Trifluoro-methanesulfonic acid 5-methyl-2-oxazol-2-yl-thiazol-4-yl ester

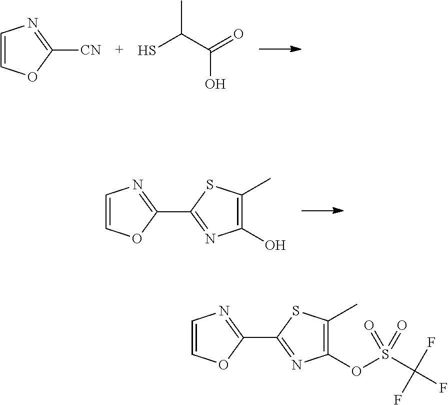

5-Methyl-2-oxazol-2-yl-thiazol-4-ol: To a mixture of 2-cyanooxazole (500 mg, 5.32 mmol) and thiolactic acid (564 mg, 5.32 mmol) was added pyridine (0.1 ml, 1.32 mmol). The mixture was heated to 100° C. for 3 h, after which it was cooled to rt, EtOH (3 ml) was added, and the suspension stirred for 10 min, filtered, and the solid dried. Further purification by column chromatography (30% EtOAc/Hexane) gave 5-Methyl-2-oxazol-2-yl-thiazol-4-ol (492 mg, 51%) as an off white solid.

Trifluoro-methanesulfonic acid 5-methyl-2-oxazol-2-yl-thiazol-4-yl ester: To a solution of 5-Methyl-2-oxazol-2-yl-thiazol-4-ol (492 mg, 2.70 mmol) in THF (35 ml) was added NaH (95 mg, 4.05 mmol) followed by N-phenyl bis(trifluoromethanesulfonimide) (1.32 g, 3.24 mmol) at 0° C. The reaction mixture was stirred at 25° C. for 1 h, at which point water was added at 0° C., and resulting solution extracted with EtOAc. The organic phase was washed with NaOH solution (0.1N), brine, then dried over Na₂SO₄, concentrated, and purified by column chromatography (8% EtOAC-Hexane) to give Trifluoro-methanesulfonic acid 5-methyl-2-oxazol-2-yl-thiazol-4-yl ester (551 mg, 65%) as a white solid.

### Intermediate 6

### Trifluoro-methanesulfonic acid 5-ethyl-2-pyridin-3-yl-thiazol-4-yl ester

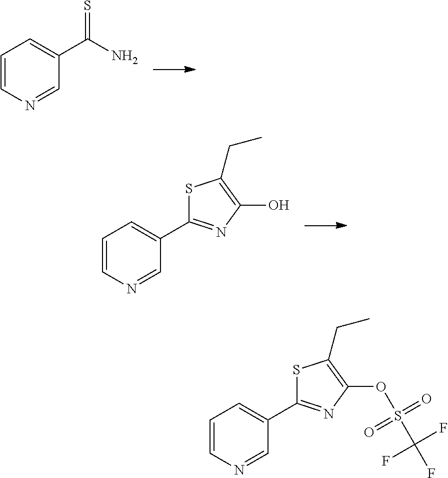 [SMILES: NC(=S)c1cccnc1 ; CCc1sc(-c2cccnc2)nc1O ; CCc1sc(-c2cccnc2)nc1OS(=O)(=O)C(F)(F)F]

Trifluoro-methanesulfonic acid 5-ethyl-2-pyridin-3-yl-thiazol-4-yl ester: To a solution of pyridine-3-carbothioamide (1 g, 7.24 mmol) in EtOH (15 mL) and pyridine (1 mL, 12.3 mmol) was added methyl 2-bromobutanoate (1 mL, 8.68 mmol). The mixture was heated at reflux for 18 hours, after which it was cooled and concentrated. The crude 5-Ethyl-2-pyridin-3-yl-thiazol-4-ol was then redissolved in DMF (36 mL) at 0° C., and to the mixture was added 60% sodium hydride (751 mg, 18.8 mmol). After stirring for 15 min at rt, 1,1,1-trifluoro-N-phenyl-N-(trifluoromethylsulfonyl)methanesulfonamide (3.87 g, 10.8 mmol) was added. The mixture was reacted for 20 min, quenched with sat. NH4Cl, diluted with diethyl ether. The mixture was washed with water, and then brine. The organic layer was concentrated, and the resulting material chromatographed (5-55% EtOAc/Hexanes to give trifluoro-methanesulfonic acid 5-ethyl-2-pyridin-3-yl-thiazol-4-yl ester (0.85 g) as an orange oil.

### Intermediate 7

### Trifluoro-methanesulfonic acid 5-methyl-2-pyrazin-2-yl-thiazol-4-yl ester

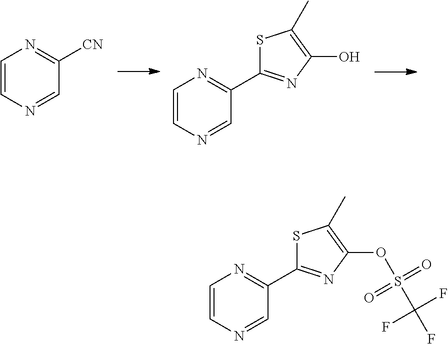 [SMILES: N#Cc1cnccn1 ; Cc1sc(-c2cnccn2)nc1O ; Cc1sc(-c2cnccn2)nc1OS(=O)(=O)C(F)(F)F]

5-Methyl-2-pyrazin-2-yl-thiazol-4-ol: In a 250 mL round-bottomed flask, pyrazine-2-carbonitrile (10 g, 95.1 mmol), pyridine (2.26 g, 2.33 ml, 28.5 mmol) and 2-mercaptopropionic acid (10.1 g, 95.1 mmol) were combined to give a light yellow solution. The reaction mixture was heated to 100° C. and stirred for 2 h. Upon cooling, the thick yellow mixture was diluted with 100 mL ethanol and stirred for 30 min. The slurry was then filtered, and washed with diethyl ether (2×100 mL) to give 5-methyl-2-pyrazin-2-yl-thiazol-4-ol (17.86 g, 97.1%) as yellow solid which was used directly without further purification.

Trifluoro-methanesulfonic acid 5-methyl-2-pyrazin-2-yl-thiazol-4-yl ester: In a 500 mL round-bottomed flask, 5-methyl-2-(pyrazin-2-yl)thiazol-4-ol (12.24 g, 63.3 mmol) was cooled to 0° C. in THF (110 ml) and stirred for 33 min. 60% sodium hydride (3.32 g, 83.0 mmol) was added followed by N-phenylbis (trifluoromethanesulfonimide) (26.6 g, 72.8 mmol) and the resultant reaction mixture was warmed to 25° C. and stirred for 1 h. The reaction mixture was poured into 50 mL H₂O and extracted with ethyl acetate (3×20 mL). The organic layers were dried over MgSO₄ and concentrated in vacuo. The crude material was purified by flash column chromatography (silica gel, 120 g, 25% to 45% ethyl acetate in hexanes) to give trifluoro-methanesulfonic acid 5-methyl-2-pyrazin-2-yl-thiazol-4-yl ester (7.45 g, 36.2%) as a colorless oil which solidified to an off-white solid.

### Intermediate 8

### 3-(5-Bromo-1-ethyl-1H-[1,2,4]triazol-3-yl)-pyridine

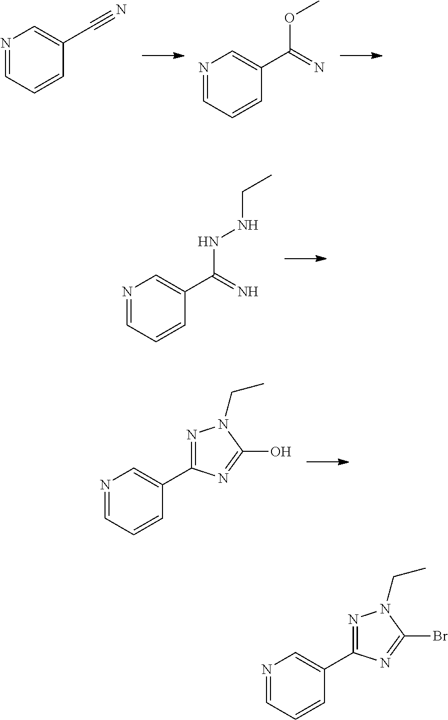

Nicotinimidic acid methyl ester: To a stirred solution of 3-cyanopyridine (5.0 g, 48.07 mmol) in methanol-1,4-dioxane (1:1; 50 ml) was added sodium methoxide (2.85 g, 52.88 mmol) at 0° C. The reaction mixture was stirred for 24 h at rt, after which the solvent was removed, and water (20 mL) was added to the resulting mass. This mixture was extracted with ethyl acetate (2×50), and the organic layers were dried, concentrated in vacuo and purified by column chromatography (20% EtOAc/Hexanes) to give nicotinimidic acid methyl ester (3.6 g, 55%) as light yellow liquid.

N′-ethylnicotinimidohydrazide: To a stirred solution of nicotinimidic acid methyl ester (2.0 g, 14.70 mmol) in dry pyridine (10 mL) was added ethyl hydrazine oxalate (2.34 g, 15.58 mmol) at rt. The mixture was stirred for 12 h, after which the solvent was removed to furnish a crude mass. This material was triturated with diethyl ether to give N′-ethylnicotinimidohydrazide (2.1 g, 87%) as a white solid.

2-Ethyl-5-pyridin-3-yl-2H-[1,2,4]triazol-3-ol: To a stirred solution of N′-ethylnicotinimidohydrazide (0.500 g, 3.05 mmol) in dry DMF (15 mL) was added CDI (0.524 g, 3.23 mmol) at rt. The mixture was then stirred for 12 h, after which the DMF was removed in vacuo, the material redissolved in methylene dichloride (25 mL), and filtered through a sintered funnel. The filtrate was concentrated under reduced pressure to provide a crude mass that was purified by column chromatography (20% methanol in DCM), to give 2-Ethyl-5-pyridin-3-yl-2H-[1,2,4]triazol-3-ol (0.200 g, 35%) as a white solid.

3-(5-Bromo-1-ethyl-1H-[1,2,4]triazol-3-yl)-pyridine: A solution of 2-Ethyl-5-pyridin-3-yl-2H-[1,2,4]triazol-3-ol (0.240 g, 1.26 mmol) in phosphorus oxybromide (1.44 g, 5.05 mmol) was stirred at 140° C. for 1 h. It was then cooled to 0° C. and the solution was basified to pH˜9 with an aqueous solution of saturated sodium bicarbonate. The aqueous mixture was extracted with ethyl acetate (3×20 mL), and the organic layers were then dried over anhydrous sodium sulfate, concentrated, and purified by column chromatography (20% EtOAc/Hexanes) to give 3-(5-Bromo-1-ethyl-1H-[1,2,4]triazol-3-yl)-pyridine (0.160 g, 50.19%) as a brown solid.

### Intermediate 9

### 3-(5-bromo-1-methyl-1H-[1,2,4]triazol-3-yl)-pyridine

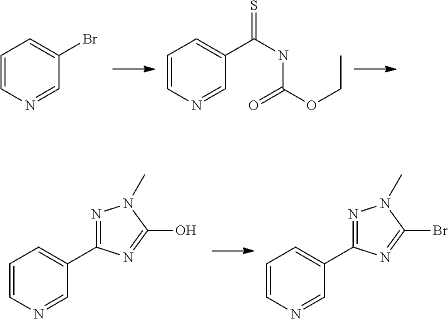

ethyl pyridine-3-carbonothioylcarbamate: n-BuLi (2.5M in THF, 60 mL, 150 mmol, 1 eq) was charged into a 3-neck 2000 ml round bottom flask, attached with a mechanical stirrer and two dropping funnels (one containing a solution of 3-bromopyridine (14.46 mL, 150 mmol, 1 eq) in 220 ml of anhydrous ether and the other one containing O-ethyl carbonisothiocyanatidate (20.4 mL, 180 mmol, 1.2 eq) in 500 mL of anhydrous THF) under argon. The solution was cooled to −78° C. The 3-bromopyridine solution was added dropwise over 45 min and stirred at −7° C. for 30 min. The solution of O-ethyl carbonisothiocyanatidate was added dropwise over 75 min. Stirring was continued and the reaction mixture was allowed to come to RT overnight. 50 mL of saturated ammonium chloride was added and the reaction mixture was concentrated to small volume, diluted with EtOAc, washed with brine, dried over anhydrous magnesium sulfated, filtered and evaporated to a red oil. Flash chromatography on silica gel (600 g) using a gradient of 0-50% EtOAc/hexanes in 60 min gave 5.2 g (16.5%) of ethyl pyridine-3-carbonothioylcarbamate as a yellow solid. LC-MS (ES) calculated for C₉H₁₀N₂O₂S, 210.26; found m/z 211.1 [M+H]⁺.

methyl-3-(pyridin-3-yl)-1H-1,2,4-triazol-5-ol: The solution of ethyl pyridine-3-carbonothioylcarbamate (4.6 g, 21.9 mmol, 1 eq) and methylhydrazine (46 mL, 873 mmol, 39.9 eq) in 46 mL THF was heated at 80° C. in an oil bath for 40 min. The reaction mixture was cooled and evaporated. Flash chromatography on silica gel (240 g) using a gradient of 20-100% EtOAc/hexanes in 60 min gave 2.65 g (69%) of 1-methyl-3-(pyridin-3-yl)-1H-1,2,4-triazol-5-ol as an off-white solid. LC-MS (ES) calculated for C₈H₈N₄O, 176.18; found m/z 177.1 [M+H]⁺.

3-(5-bromo-1-methyl-1H-[1,2,4]triazol-3-yl)-pyridine: 1-methyl-3-(pyridin-3-yl)-1H-1,2,4-triazol-5-ol (1.2 g, 11.33 mmol, 1 eq) and phosphoryl tribromide (14.56 g, 50.84 mmol, 3.98 eq) were combined in a microwave reaction vessel and sealed. The mixture was heated at 120° C. in an oil bath for 2 hrs. The reaction mixture was cooled in acetone/dry ice bath and neutralized carefully with a saturated sodium bicarbonate solution, extracted with EtOAc, dried over anhydrous magnesium, filtered and evaporated. Flash chromatography on silica gel (120 g) using a gradient column of 0-60% EtOAc/hexane in 45 min gave 2.28 g (74%) of 3-(5-bromo-1-methyl-1H-[1,2,4]triazol-3-yl)-pyridine as a white solid. LC-MS (ES) calculated for C₈H₇BrN₄, 239.08; found m/z 240.0 [M+H]⁺.

### Intermediate 10

### 5-Bromo-4-methyl-pyridine-2-carboxylic acid methylamide

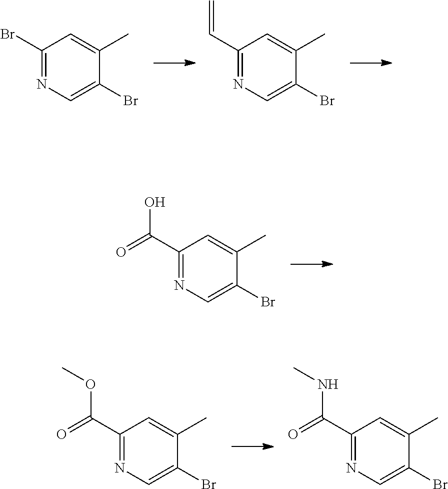

5-Bromo-4-methyl-2-vinyl-pyridine: To a solution of 2,5-Dibromo-4-methyl-pyridine (10 g, 39.8 mmol) and trivinyl cyclotriboroxane (6.44 g, 39.8 mmol) in DME (150 ml) was added K₂CO₃ (5.5 gm, 39.8 mmol) in water (30 mL) followed by Pd(PPh₃)₄ (460 mg, 0.398 mmol). The mixture was stirred at 100° C. for 4 h, after which it was filtered through Celite. The filtrate was diluted with water and extracted with EtOAc. The organic phase was washed with brine, dried, concentrated, and the crude material was purified by column chromatograph to give 5-Bromo-4-methyl-2-vinyl-pyridine (7.04 gm, 70%) as light yellow solid.

5-Bromo-4-methyl-pyridine-2-carboxylic acid: To a solution of 5-Bromo-4-methyl-2-vinyl-pyridine (600 mg, 3 mmol) in acetone-water (1:1, 54 ml) was added KMnO₄ (957 mg, 6 mmol). The mixture was stirred for 3 days at rt, at which point it was filtered, concentrated, and purified by column chromatograph to give 5-Bromo-4-methyl-pyridine-2-carboxylic acid (700 mg, 92%) as white solid.

5-Bromo-4-methyl-pyridine-2-carboxylic acid methyl ester: To a solution of 5-Bromo-4-methyl-pyridine-2-carboxylic acid (650 mg, 3.0 mmol) in MeOH (2 ml) was added conc. H₂SO₄ (0.06 ml). The mixture was refluxed for 14 h, after which it was cooled to 0° C., neutralized with saturated NaHCO₃, filtered, concentrated, and purified by column chromatography to give 5-Bromo-4-methyl-pyridine-2-carboxylic acid methyl ester (340 mg, 49%) as white solid.

5-Bromo-4-methyl-pyridine-2-carboxylic acid methylamide: To 5-Bromo-4-methyl-pyridine-2-carboxylic acid methyl ester (200 mg, 0.869 mmol) and methylamine (135 mg, 11.34 mmol) was added (CH₃)₃Al (0.6 mg, 0.008 mmol). The mixture was placed in a sealed tube and heated at 100° C. for 1 h, after which the mixture was cooled, quenched with water, and extracted with EtOAc. The organic phase was dried, concentrated, and purified by column chromatograph to give 5-Bromo-4-methyl-pyridine-2-carboxylic acid methylamide (130 mg, 65%) as an off-white solid.

### Intermediate 11

### 5-Bromo-4-methyl-pyridine-2-carboxylic acid (2-hydroxy-ethyl)-amide

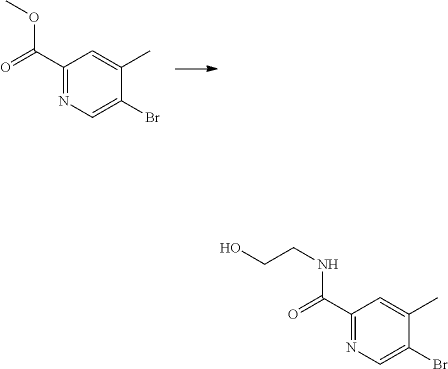

5-Bromo-4-methyl-pyridine-2-carboxylic acid (2-hydroxy-ethyl)-amide: To 5-bromo-4-methyl-pyridine-2-carboxylic acid methyl ester (200 mg, 0.869 mmol) and 2-amino-ethanol (265 mg, 4.34 mmol) was added (CH₃)₃Al (0.6 mg, 0.008 mmol). The mixture was placed in a sealed tube and heated at 100° C. for 1 h, after which the mixture was cooled, quenched with water, and extracted with EtOAc. The organic phase was dried, concentrated, and purified by column chromatograph to give 5-Bromo-4-methyl-pyridine-2-carboxylic acid (2-hydroxy-ethyl)-amide (130 mg, 65%) as an off-white solid.

### Intermediate 12

### Trifluoro-methanesulfonic acid 2-ethyl-5-pyrazin-2-yl-2H-pyrazol-3-yl ester

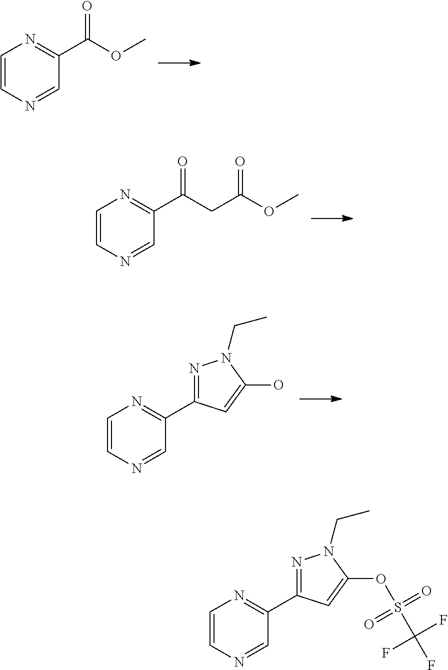

Methyl 3-oxo-3-(pyrazin-2-yl)propanoate: To a stirred solution of sodium methoxide (25% in MeOH, 27.54 mL, 72.4 mmol, 1 eq) in 90 mL of toluene at 110° C. in a 3-neck flask attached with a mechanical stirrer, condenser and dropping funnel was added a solution of methylpyrazine-2-carboxylate (10 g, 72.4 mmol, 1 eq) in 115 mL of methyl acetate, dropwise, over a period of ˜35-40 min. A yellow precipitate was formed. Stirring was continued at 110° C. for 3 hrs. The reaction was cooled and the yellow precipitate was filtered and washed with a small quantity of toluene. This solid was taken into 200 mL of saturated ammonium chloride and 400 mL of EtOAc. The aqueous layer was extracted twice with EtOAc. The combined organic layers were dried over magnesium sulfate, filtered and evaporated to give 6.52 g (50%) of methyl 3-oxo-3-(pyrazin-2-yl)propanoate as a yellow solid.

Ethyl-3-(pyrazin-2-yl)-1H-pyrazol-5-ol: Ethylhydrazine oxalate (6.89 g, 45.9 mmol, 1 eq) was stirred with 450 mL of anhydrous ethanol for 10 min. To this was added methyl 3-oxo-3-(pyrazin-2-yl)propanoate (8.27 g, 45.9 mmol, 1 eq) and the mixture was refluxed for 10 hrs. The reaction was cooled, evaporated, taken into 300 ml of EtOAc, extracted with water and brine, dried over anhydrous magnesium, filtered and evaporated to yield 8.7 g of 1-ethyl-3-(pyrazin-2-yl)-1H-pyrazol-5-ol as a red oil. This material was used without further purification.

Trifluoro-methanesulfonic acid 2-ethyl-5-pyrazin-2-yl-2H-pyrazol-3-yl ester: To a stirred solution of 1-ethyl-3-(pyrazin-2-yl)-1H-pyrazol-5-ol (8.7 g, 45.7 mmol, 1 eq) in 230 mL DMF at 0° C. was added NaH (2.93 g, 73.2 mmol, 1.6 eq). The mixture was allowed to warm to rt and stirred for 1 hr. 1,1,1-Trifluoro-N-phenyl-N-(trifluoromethylsulfonyl)methanesulfonamide (24.5 g, 68.6 mmol, 1.5 eq) was added and stirred at RT for 90 min. The mixture was cooled in an ice bath, quenched with saturated ammonium chloride, evaporated and taken into EtOAc, extracted with water and brine, dried over anhydrous magnesium sulfate, filtered and evaporated to an oil. Flash chromatography on silica gel (400 g) using a gradient of 10-30% EtOAC/hexane gave 9.27 g (62.9%) of trifluoro-methanesulfonic acid 2-ethyl-5-pyrazin-2-yl-2H-pyrazol-3-yl ester as a white solid. LC-MS (ES) calculated for C₁₀H₉F₃N₄O₃S, 322.27; found m/z 322.9 [M+H]⁺.

### Intermediate 13

### 5-Bromo-2-methanesulfonyl-4-methyl-pyridine

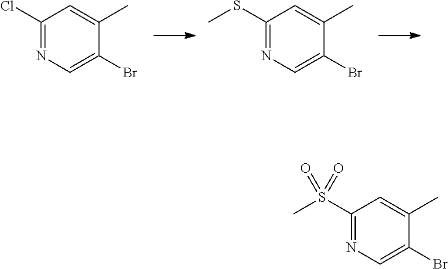

5-Bromo-4-methyl-2-methylsulfanyl-pyridine: A mixture of 5-bromo-2-chloro-4-methylpyridine (1.81 g, 8.8 mmol), and sodium thiomethoxide (0.68 g, 9.8 mmol) in 10 mL of dioxane was placed in a 110° C. oil bath for 3 hrs., cooled and extracted between ethyl acetate and water, washed organic layer with water, dried over sodium sulfate, filtered and concentrated to give the crude product as a pale-yellow liquid (1.83 g). The crude product was carried onto the oxidation step without further purification.

5-Bromo-2-methanesulfonyl-4-methyl-pyridine: To a 0° C. solution of 5-bromo-4-methyl-2-(methylthio)pyridine (1.83 g, 8.4 mmol) in 25 mL of dichloromethane was added MCPBA (3.50 g, 55% pure, 11 mmol). The reaction mixture was stirred for 1 hr., partitioned between water and dichloromethane, then washed the organic layer twice with aq. sodium bicarbonate, dried over sodium sulfate, filtered and concentrated to give a crude yellow solid. The crude mixture was loaded onto Si-gel and purified by flash chromatography (20:80-1:1 ethyl acetate/hexanes then 100% ethyl acetate) to give the product as a light-yellow solid (0.64 g, 29% over two steps). MS (M+H)=252.

### Intermediate 14

### 1-chloro-2-ethynyl-3-fluoro-benzene

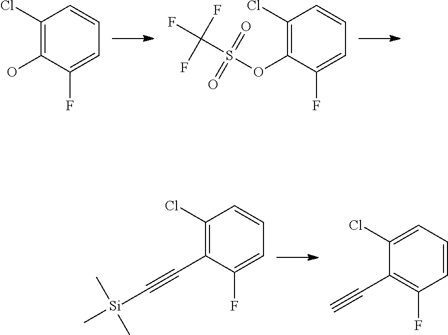

2-chloro-6-fluorophenyl trifluoromethanesulfonate: To a stirred solution of pyridine (26.7 mL, 207 mmol, 1 eq) and 2-chloro-6-fluorophenol (30.3 g, 207 mmol, 1 eq) in methylene chloride (380 mL) at 0° C. was added trifluoromethanesulfonic anhydride (45.2 mL, 207 mmol, 1 eq) dropwise. The mixture was stirred at RT for 3 hrs, evaporated, dissolved in EtOAc, washed with water and brine, dried over anhydrous magnesium sulfate, filtered and evaporated to yield 2-chloro-6-fluorophenyl trifluoromethanesulfonate as a yellow oil that was used without purification.

(2-chloro-6-fluoro-phenylethynyl)-trimethyl-silane: To a solution of 2-chloro-6-fluorophenyl trifluoromethanesulfonate (10 g, 35.9 mmol, 1 eq), ethynyltrimethylsilane (5.29 g, 53.8 mmol, 1.5 eq) and triethylamine (5.45 g, 53.8 mmol, 1.5 eq) in anhydrous acetonitrile (200 mL) was added bis(triphenylphosphine)palladium (II) chloride (500 mg, 0.717 mmol, 0.02 eq). The reaction mixture was heated to reflux under argon for 20 h, cooled, evaporated, and the residue redissolved in 300 ml hexanes and stirred for 20 min. It was then washed with water and brine and dried over anhydrous magnesium sulfate, filtered, evaporated to dryness, and chromatographed (hexanes) to give (2-chloro-6-fluoro-phenylethynyl)-trimethyl-silane (6.4 g, 79%) as a solid.

chloro-2-ethynyl-3-fluoro-benzene: To a solution of ((2-chloro-6-fluorophenyl)ethynyl)trimethylsilane (1.0 g, 4.41 mmol, 1 eq) in MeOH (40 ml) was added potassium carbonate (0.616 gm, 4.41 mmol, 1 eq). The reaction mixture was stirred at rt for 3 hrs, diluted with dichloromethane and water and separated. The organic layer was dried over anhydrous magnesium sulfate and evaporated to yield 580 mg (85%) of 1-chloro-2-ethynyl-3-fluoro-benzene as a dark oil that was used without further purification.

### Part II: Preparation of Certain Embodiments of the Invention

### Example 1

### 4-[6-(2-chloro-6-fluoro-phenyl)-5H-pyrrolo[3,2-d]pyrimidin-2-yl]-3,N,N-trimethyl-benzenesulfonamide

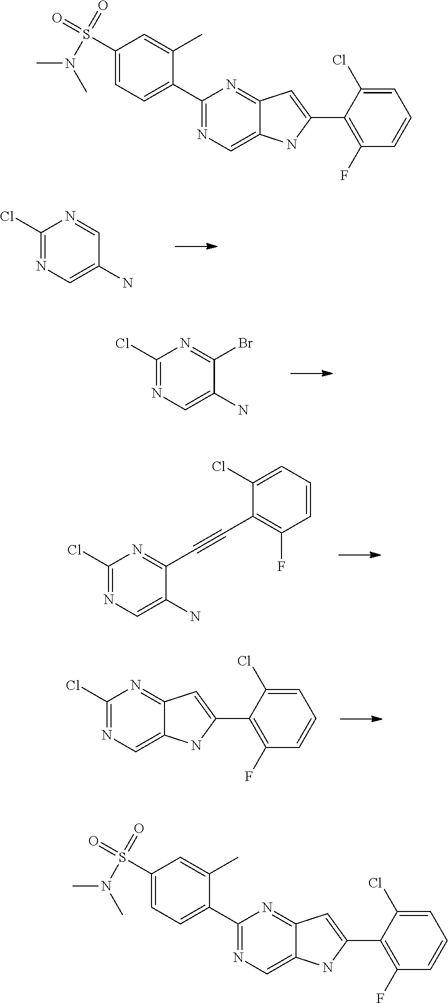 [SMILES: Cc1cc(S(=O)(=O)N(C)C)ccc1-c1ncc2[nH]c(-c3c(F)cccc3Cl)cc2n1 ; Nc1cnc(Cl)nc1 ; Nc1cnc(Cl)nc1Br ; Nc1cnc(Cl)nc1C#Cc1c(F)cccc1Cl ; Fc1cccc(Cl)c1-c1cc2nc(Cl)ncc2[nH]1 ; Cc1cc(S(=O)(=O)N(C)C)ccc1-c1ncc2[nH]c(-c3c(F)cccc3Cl)cc2n1]

4-Bromo-2-chloro-pyrimidin-5-ylamine: To a solution of 2-chloropyrimidin-5-amine (500 mg, 3.86 mmol, 1 eq) and powdered iron (10.8 mg, 0.193 mmol, 0.05 eq) in dichloromethane (30 ml) at 0° C. was added dropwise a solution of bromine (617 mg, 2.86 mmol, 1 eq) in dichloromethane (10 mL). The reaction mixture was allowed to come to room temperature slowly and stirred overnight (23 h). The iron was filtered off, washed with a small amount of dichloromethane and the organic layer evaporated to dryness to give 4-bromo-2-chloro-pyrimidin-5-ylamine (contaminated with a small amount of starting material) that was used without purification in the next step.

2-chloro-4-(2-chloro-6-fluoro-phenylethynyl)-pyrimidin-5-ylamine: To a solution of 4-bromo-2-chloropyrimidin-5-amine (193 mg, 0.926 mmol, 1 eq) and 1-chloro-2-ethynyl-3-fluorobenzene (285 mg, 184 mmol, 1.99 eq) in TEA (10 mL) was added copper(I) iodide (17.6 mg, 0.0927 mmol, 0.1 eq) and tetrakis(triphenylphosphine)palladium(0), (160 mg, 0.139 mmol, 0.15 eq). The mixture was heated at 80° C. for 8 hrs, then cooled and evaporated. To the residue was added EtOAC and water. The organic layer was separated, washed with brine, dried over anhydrous magnesium sulfate, evaporated, and purified by flash chromatography (0% to 15% EtOAc/hexanes) to give 2-chloro-4-(2-chloro-6-fluoro-phenylethynyl)-pyrimidin-5-ylamine (161 mg, 62%) as a yellow solid.

2-chloro-6-(2-chloro-6-fluoro-phenyl)-5H-pyrrolo[3,2-d]pyrimidine: To a solution of 2-chloro-4-((2-chloro-6-fluorophenyl)ethynyl)pyrimidin-5-amine (155 mg, 0.549 mmol, 1 eq) in NMP (10 mL) was added a solution of potassium t-butoxide (184 mg, 1.648 mmol, 3 eq) in NMP (10 mL). The reaction mixture was heated at 55° C. for 30 min, after which it was cooled in an ice bath, and the pH adjusted to 7 by addition of pH 7 buffer. EtOAc was then added and the organic layer was separated, washed with brine, dried over anhydrous magnesium sulfate, evaporated, and purified by flash chromatography (0% to 15% EtOAc/hexanes) to give of 2-chloro-6-(2-chloro-6-fluoro-phenyl)-5H-pyrrolo[3,2-d]pyrimidine (62 mg, 40%).

4-[6-(2-chloro-6-fluoro-phenyl)-5H-pyrrolo[3,2-d]pyrimidin-2-yl]-3,N,N-trimethyl-benzenesulfonamide: To a solution of 2-chloro-6-(2-chloro-6-fluorophenyl)-5H-pyrrolo[3,2-d]pyrimidine (20 mg, 0.0707 mmol, 0.5 eq) and 4-(N,N-dimethylsulfamoyl)-2-methylphenylboronic acid (34 mg, 0.140 mmol, 1 eq) in dioxane (1.8 ml) and water (0.2 ml) was added potassium carbonate (19.6 mg, 0.141 mmol, 2 eq) and [1,1′-bis(diphenylphospheno)ferrocene]dichloropalladium(II) (10.2 mg, 0.014 mmol, 0.1 eq). The mixture was heated at 110° C. for 8 hrs, then cooled and evaporated. To the residue was added EtOAc and water. The organic layer was separated, washed with brine, dried over anhydrous magnesium sulfate, evaporated, and purified by flash chromatography (0% to 25% EtOAc/hexanes) to give 4-[6-(2-chloro-6-fluoro-phenyl)-5H-pyrrolo[3,2-d]pyrimidin-2-yl]-3,N,N-trimethyl-benzenesulfonamide (13 mg, 21%) as a white solid. MS: 445 (M+H).

### Example 2

### 6-(2-Chloro-phenyl)-2-(2-methyl-5-trifluoromethyl-2H-pyrazol-3-yl)-5H-pyrrolo[2,3-b]pyrazine

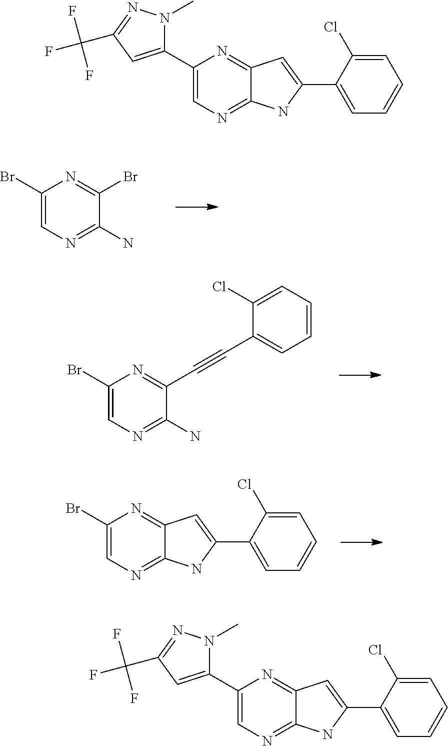 [SMILES: Cn1nc(C(F)(F)F)cc1-c1cnc2[nH]c(-c3ccccc3Cl)cc2n1 ; Nc1ncc(Br)nc1Br ; Nc1ncc(Br)nc1C#Cc1ccccc1Cl ; Clc1ccccc1-c1cc2nc(Br)cnc2[nH]1 ; Cn1nc(C(F)(F)F)cc1-c1cnc2[nH]c(-c3ccccc3Cl)cc2n1]

5-Bromo-3-(2-chloro-phenylethynyl)-pyrazin-2-ylamine: To a solution of 2-amino-3,5-dibromopyrazine (1 g, 3.95 mmol, 1 eq) and 2-chlorophenylacetylene (0.540 g, 3.954 mmol, 1 eq) in DMF (16 mL) were added PdCl₂(PPh₃)₂ (0.135 g, 0.198 mmol, 5 mol %), CuI (0.190 g, 0.395 mmol, 0.10 eq), and TEA (2.40 g, 3.29 mL, 23.7 mmol, 6 eq). The mixture was stirred at rt for 15 min, at which point the TEA was removed in vacuo, the reaction mixture was diluted with water, extracted ethyl acetate, washed with water then brine, and dried over magnesium sulfate. After filtration, the solvent was concentrated in vacuo, and the residue chromatographed twice (20-40% EtOAc/hexanes) to give 5-Bromo-3-(2-chloro-phenylethynyl)-pyrazin-2-ylamine (˜390 mg, containing approximately 10% bis-coupled material) as a yellow solid.

2-Bromo-6-(2-chloro-phenyl)-5H-pyrrolo[2,3-b]pyrazine: To solution of 5-Bromo-3-(2-chloro-phenylethynyl)-pyrazin-2-ylamine (0.144 g, 0.467 mmol, 1 eq) in NMP (2 mL) was added potassium tert-butoxide (0.157 g, 1.40 mmol, 3 eq). The reaction mixture immediately turned a deep red and was then heated to 75° C. for 2.5 h. Upon completion as deemed by ¹H NMR, the mixture was cooled, diluted with sat. aq. ammonium chloride, extracted 1:2 ethyl acetate/ether (3×), washed brine, and dried over magnesium sulfate. The organic layer was concentrated to give a solid that was triturated with Et₂O to give 2-bromo-6-(2-chloro-phenyl)-5H-pyrrolo[2,3-b]pyrazine (75 mg, 52%) as a peach colored solid.

### 6-(2-Chloro-phenyl)-2-(2-methyl-5-trifluoromethyl-2H-pyrazol-3-yl)-5H-pyrrolo[2,3-b]pyrazine

To a suspension of 2-bromo-6-(2-chloro-phenyl)-5H-pyrrolo[2,3-b]pyrazine (0.075 g, 0.243 mmol, 1 eq) and 1-methyl-3-trifluoromethylpyrazole-5-boronic acid (0.061 g, 0.316 mmol, 1.3 eq) in dioxane/H₂O (1.5 mL/0.45 mL) was added [1,1′-bis(diphenylphospheno)ferrocene]dichloropalladium(II) methylene chloride complex (0.020 g, 0.024 mmol, 0.10 eq) and K₂CO₃ (0.101 g, 0.729 mmol, 3 eq). The mixture was heated to 110° C. for 18 h, cooled, and filtered through a pad of Celite that was then washed with EtOAc. The filtrate was then washed with water, brine, dried (MgSO₄), concentrated in vacuo, and chromatographed (20% EtOAc/Hexanes) to give 6-(2-Chloro-phenyl)-2-(2-methyl-5-trifluoromethyl-2H-pyrazol-3-yl)-5H-pyrrolo[2,3-b]pyrazine (0.010 g, 12%) as a white solid. MS: 378 (M+H).

### Example 3

### 4-[6-(2-Chloro-phenyl)-5H-pyrrolo[2,3-b]pyrazin-2-yl]-3-methyl-benzoic acid methyl ester

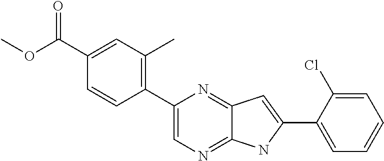 [SMILES: COC(=O)c1ccc(-c2cnc3[nH]c(-c4ccccc4Cl)cc3n2)c(C)c1]

4-[6-(2-Chloro-phenyl)-5H-pyrrolo[2,3-b]pyrazin-2-yl]-3-methyl-benzoic acid methyl ester: Was prepared from 2-bromo-6-(2-chloro-phenyl)-5H-pyrrolo[2,3-b]pyrazine and 4-(Methoxycarbonyl)-2-methylphenylboronic acid pinacol ester in a manner identical to that described in Example 2, to give 60 mg as an off-white solid. MS: 378 (M+H).

### Example 4

### 4-(6-(2-Chlorophenyl)-5H-pyrrolo[2,3-b]pyrazin-2-yl)-N,N,3-trimethylbenzenesulfonamide

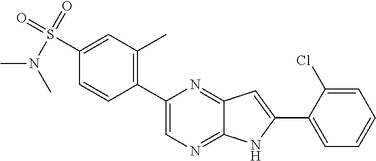 [SMILES: Cc1cc(S(=O)(=O)N(C)C)ccc1-c1cnc2[nH]c(-c3ccccc3Cl)cc2n1]

### 4-(6-(2-Chlorophenyl)-5H-pyrrolo[2,3-b]pyrazin-2-yl)-N,N,3-trimethylbenzenesulfonamide

Was prepared from 2-bromo-6-(2-chloro-phenyl)-5H-pyrrolo[2,3-b]pyrazine and 4-(N,N-dimethylsulfamoyl)-2-methylphenylboronic acid in a manner identical to that described in Example 2, to give 52 mg as a light yellow solid. MS: 427 (M+H).

### Example 5

### 6-(2-chloro-6-fluorophenyl)-2-(1-methyl-3-(trifluoromethyl)-1H-pyrazol-5-yl)-5H-pyrrolo[2,3-b]pyrazine

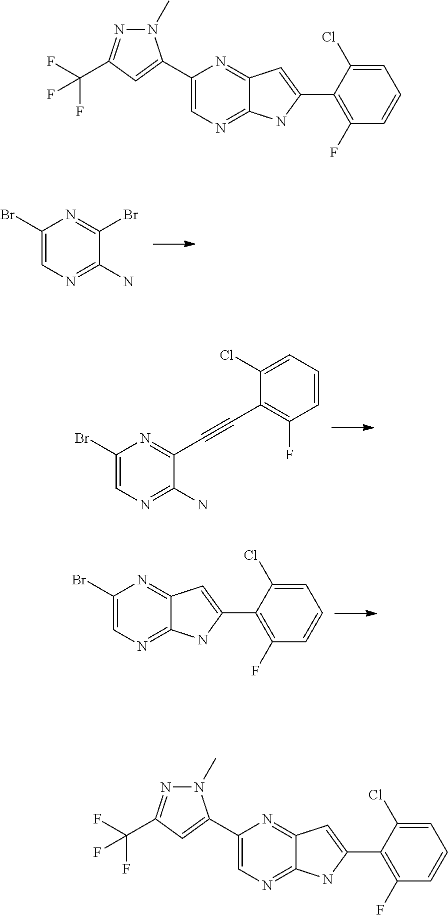 [SMILES: Cn1nc(C(F)(F)F)cc1-c1cnc2[nH]c(-c3c(F)cccc3Cl)cc2n1 ; Nc1ncc(Br)nc1Br ; Nc1ncc(Br)nc1C#Cc1c(F)cccc1Cl ; Fc1cccc(Cl)c1-c1cc2nc(Br)cnc2[nH]1 ; Cn1nc(C(F)(F)F)cc1-c1cnc2[nH]c(-c3c(F)cccc3Cl)cc2n1]

5-Bromo-3-(2-chloro-6-fluoro-phenylethynyl)-pyrazin-2-ylamine: To a solution of 2-amino-3,5-dibromopyrazine (1.6 g, 6.33 mmol) and (2-chloro-6-fluoro-phenylethynyl)-trimethyl-silane (2.15 g, 9.49 mmol) in DMF (25 mL) was added copper iodide (120 mg, 0.63 mmol) and tetrakis(triphenylphosphine)palladium(0) (366 mg, 0.32 mmol), followed by TEA (12.7 mL, 6.33 mmol). The reaction mixture was heated to 110° C. for 18 h, cooled, and the TEA was removed in vacuo. The mixture was then diluted with water, extracted diethyl ether, washed with water then brine, and dried over magnesium sulfate. After filtration, the solvent was concentrated in vacuo, and the residue chromatographed (20% to 70% EtOAc/hexane) to give 5-bromo-3-(2-chloro-6-fluoro-phenylethynyl)-pyrazin-2-ylamine (˜900 mg contaminated with 50% 2-amino-3,5-dibromopyrazine which was suitable for use in the next reaction).

2-Bromo-6-(2-chloro-6-fluoro-phenyl)-5H-pyrrolo[2,3-b]pyrazine: To a solution of 5-bromo-3-(2-chloro-6-fluoro-phenylethynyl)-pyrazin-2-ylamine (˜800 mg, 2.45 mmol, Eq: 1.00) in NMP (15 ml) was added potassium tert-butoxide (825 mg, 7.35 mmol, Eq: 3.0). The reaction mixture immediately turned dark and was then heated to 75° C. for 2 h. After cooling, the mixture was diluted with sat. aq. ammonium chloride, extracted with diethyl ether, washed with water then brine, and dried over magnesium sulfate. After filtration the solvents were removed in vacuo and the residue chromatographed (33% to 77% ethyl acetate/hexanes) to give 2-bromo-6-(2-chloro-6-fluoro-phenyl)-5H-pyrrolo[2,3-b]pyrazine (˜90 mg contaminated with an unknown impurity but suitable for use in the next reaction).

6-(2-chloro-6-fluorophenyl)-2-(1-methyl-3-(trifluoromethyl)-1H-pyrazol-5-yl)-5H-pyrrolo[2,3-b]pyrazine: To a solution 2-bromo-6-(2-chloro-6-fluorophenyl)-5H-pyrrolo[2,3-b]pyrazine (55 mg, 168 μmol, Eq: 1.00) and 1-methyl-3-trifluoromethylpyrazole-5-boronic acid (39.2 mg, 202 μmol, Eq: 1.2) in dioxane (3.00 ml) and water (0.8 ml) was added 1,1′-bis(diphenylphosphino)ferrocenedichloro palladium(II) (12.3 mg, 16.8 μmol, Eq: 0.1) and potassium carbonate (69.8 mg, 505 μmol, Eq: 3.0). The mixture was heated to 110° C. for 4 h, cooled, and filtered through a pad of Celite that was then washed with DCM. After the solvent was removed in vacuo, the residue was redissolved in DCM, washed with water, brine, dried (MgSO₄), concentrated in vacuo, and chromatographed (20% EtOAc-Hexane) to give 6-(2-chloro-6-fluorophenyl)-2-(1-methyl-3-(trifluoromethyl)-1H-pyrazol-5-yl)-5H-pyrrolo[2,3-b]pyrazine (31 mg, 47% yield). MS: 396 (M+H).

### Example 6

### 6-Cyclohexyl-2-(1-methyl-3-(trifluoromethyl)-1H-pyrazol-5-yl)-5H-pyrrolo[2,3-b]pyrazine

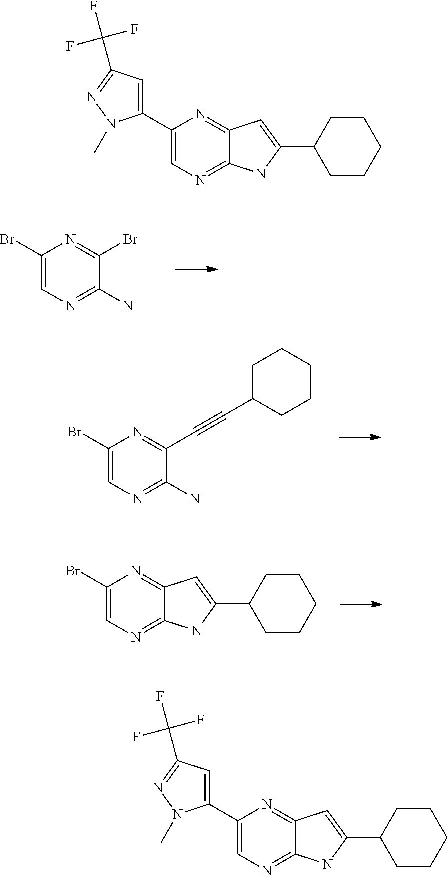 [SMILES: Cn1nc(C(F)(F)F)cc1-c1cnc2[nH]c(C3CCCCC3)cc2n1 ; Nc1ncc(Br)nc1Br ; Nc1ncc(Br)nc1C#CC1CCCCC1 ; Brc1cnc2[nH]c(C3CCCCC3)cc2n1 ; Cn1nc(C(F)(F)F)cc1-c1cnc2[nH]c(C3CCCCC3)cc2n1]

5-Bromo-3-cyclohexylethynyl-pyrazin-2-ylamine: To a solution of 2-amino-3,5-dibromopyrazine (1.5 g, 5.93 mmol) and ethynylcyclohexane (642 mg, 5.93 mmol) in THF (24 mL) was added copper iodide (113 mg, 0.59 mmol) and tetrakis(triphenylphosphine)palladium(0) (343 mg, 0.30 mmol), followed by TEA (11.9 mL, 5.93 mmol). The reaction mixture was heated to 80° C. for 4 h, cooled, and the TEA was removed in vacuo. The mixture was then diluted with water, extracted ethyl acetate, washed with water then brine, and dried over magnesium sulfate. After filtration, the solvent was concentrated in vacuo, and the residue chromatographed (5% to 33% EtOAc/hexane) to give 5-bromo-3-cyclohexylethynyl-pyrazin-2-ylamine (˜1.65 g, contaminated with a small amount of triphenylphosphine and/or triphenylphosphine oxide and suitable for use in the next reaction).

2-Bromo-6-cyclohexyl-5H-pyrrolo[2,3-b]pyrazine: To a solution of 5-bromo-3-cyclohexylethynyl-pyrazin-2-ylamine (˜1.65 g, 5.0 mmol, Eq: 1.00) in NMP (30 ml) was added potassium tert-butoxide (1.98 g, 17.7 mmol, Eq: 3.0). The reaction mixture immediately turned a deep red and was then heated to 75° C. for 4 h. After cooling, the mixture was diluted with sat. aq. ammonium chloride, extracted with diethyl ether, washed with brine, and dried over magnesium sulfate. After filtration the solvents were removed in vacuo and the residue chromatographed (15% to 50% ethyl acetate/hexanes gradient) to give 2-bromo-6-cyclohexyl-5H-pyrrolo[2,3-b]pyrazine (950 mg, 58% yield).

6-Cyclohexyl-2-(1-methyl-3-(trifluoromethyl)-1H-pyrazol-5-yl)-5H-pyrrolo[2,3-b]pyrazine: To a solution of 2-bromo-6-cyclohexyl-5H-pyrrolo[2,3-b]pyrazine (100 mg, 357 μmol, Eq: 1.00) and 1-methyl-3-(trifluoromethyl)-1H-pyrazol-5-ylboronic acid (90.0 mg, 464 mmol, Eq: 1.3) in dioxane (6.35 ml) and water (1.59 ml) was added tetrakis(triphenylphosphine)palladium(0) (41.2 mg, 35.7 μmol, Eq: 0.1) and potassium carbonate (148 mg, 1.07 mmol, Eq: 3). The mixture was heated at 95° C. for 2 h, before being concentrated onto silica gel and chromatographed directly (7-23% ethyl acetate/hexane gradient) to give 6-cyclohexyl-2-(1-methyl-3-(trifluoromethyl)-1H-pyrazol-5-yl)-5H-pyrrolo[2,3-b]pyrazine (91 mg, 73%) as an off-white solid. MS: 350 (M+H).

### Example 7

### 4-(6-cyclohexyl-5H-pyrrolo[2,3-b]pyrazin-2-yl)-N,N,3-trimethylbenzenesulfonamide

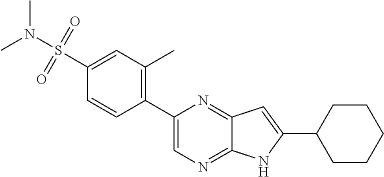 [SMILES: Cc1cc(S(=O)(=O)N(C)C)ccc1-c1cnc2[nH]c(C3CCCCC3)cc2n1]

### 4-(6-cyclohexyl-5H-pyrrolo[2,3-b]pyrazin-2-yl)-N,N,3-trimethylbenzenesulfonamide

Was prepared from 2-bromo-6-cyclohexyl-5H-pyrrolo[2,3-b]pyrazine and 4-(N,N-dimethylsulfamoyl)-2-methylphenylboronic acid in a manner identical to that described in Example 6, to give 60 mg as a white solid. MS: 399 (M+H).

### Example 8

### Jurkat IL-2 Production Assay

Cell:

Jurkat cell (ATCC) was grown in RPMI 1640 with 10% FBS and 1% penicillin/streptomycin. The cell density was kept at 1.2˜1.8×10⁶/mL in culture flask before seeding into culture plate, and the cell density in the plate was 0.5×10⁶/2004/well.

Culture Media:

RPMI 1640 with 1% FBS or 30% FBS for high serum assay.

Test Compound:

serial dilution was done in 100% DMSO, and intermediate dilution was done with RPMI 1640 medium with 1% FBS. The DMSO final concentration in culture well was 0.25%.

Stimulant:

PHA (Sigma#L9017-10MG) was used for the assay with 1% FBS in culture medium, and added after 10 minutes exposure of cell to compound/DMSO. The PHA final concentration in culture well was 5 μg/mL. PMA (Sigma# P-8139 5MG)/Ionomycin (Sigma#10634-5MG) was used for the assay with 30% FBS in culture medium, and added at same time point as the 1% FBS culture assay. The final concentration of PMA was 50 ng/mL, and Ionomycin final concentration was 500 ng/mL.

Incubation:

at 37° C. with 5% CO₂ and 95% humidity for 18 h˜20 h.

IC50:

IC50 was calculated with the data analysis software XLfit4, General Pharmacology model 251.

Using the above procedure, the IC₅₀ values for certain embodiments of the invention are provided in Table 1:

TABLE 1

| Example Number | IC₅₀ (μM) |
|---|---|
| 1 | 0.687 |
| 2 | 0.766 |
| 3 | 0.205 |
| 4 | 0.133 |
| 5 | 0.269 |
| 6 | 0.681 |
| 7 | 0.184 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

1. A compound of Formula (I): 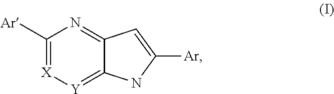
wherein:
one of X or Y is C and the other is N;
Ar is unsubstituted cycloalkyl, unsubstituted phenyl or phenyl mono- or bi-substituted independently with halogen;
Ar′ is—phenyl, unsubstituted or mono- or bi-substituted independently with —SO₂N(CH₃)₂, lower alkyl or —C(O)OCH₃, or
heteroaryl, unsubstituted or mono- or bi-substituted independently with lower alkyl or haloalkyl;
or a pharmaceutically acceptable salt thereof.

2. The compound according to claim 1, wherein X is C and Y is N.

3. The compound according to claim 1, wherein X is N and Y is C.

4. The compound according to claim 1, wherein Ar is phenyl mono- or bi-substituted independently with halogen, and Ar′ is phenyl, unsubstituted or mono- or bi-substituted independently with —SO₂N(CH₃)₂, lower alkyl or —C(O)OCH₃.

5. The compound according to claim 1, wherein Ar is phenyl mono- or bi-substituted independently with halogen, and Ar′ is heteroaryl, unsubstituted or mono- or bi-substituted independently with lower alkyl or haloalkyl.

6. The compound according to claim 1, wherein is Ar cyclohexyl and Ar′ is phenyl, unsubstituted or mono- or bi-substituted independently with —SO₂N(CH₃)₂, lower alkyl or —C(O)OCH₃.

7. The compound according to claim 1, wherein is Ar is cyclohexyl and Ar′ is heteroaryl, unsubstituted or mono- or bi-substituted independently with lower alkyl or haloalkyl.

8. The compound according to claim 1, wherein Ar is phenyl bisubstitued independently with chlorine or fluorine.

9. The compound according to claim 1, wherein Ar is cyclohexyl.

10. The compound according to claim 1, wherein Ar′ is N,N-trimethyl-benzenesulfonamide or methyl-benzoic acid methyl ester.

11. The compound according to claim 1, wherein Ar′ is pyrazolyl, unsubstituted or mono- or bi-substituted independently with methyl or —CF₃.

12. The compound according to claim 1, wherein said compound is:
4-[6-(2-chloro-6-fluoro-phenyl)-5H-pyrrolo[3,2-d]pyrimidin-2-yl]-3,N,N-trimethyl-benzenesulfonamide;
6-(2-Chloro-phenyl)-2-(2-methyl-5-trifluoromethyl-2H-pyrazol-3-yl)-5H-pyrrolo[2,3-b]pyrazine;
4-[6-(2-Chloro-phenyl)-5H-pyrrolo[2,3-b]pyrazin-2-yl]-3-methyl-benzoic acid methyl ester;
4-(6-(2-Chlorophenyl)-5H-pyrrolo[2,3-b]pyrazin-2-yl)-N,N,3-trimethylbenzenesulfonamide;
6-(2-chloro-6-fluorophenyl)-2-(1-methyl-3-(trifluoromethyl)-1H-pyrazol-5-yl)-5H-pyrrolo[2,3-b]pyrazine;
6-Cyclohexyl-2-(1-methyl-3-(trifluoromethyl)-1H-pyrazol-5-yl)-5H-pyrrolo[2,3-b]pyrazine; or
4-(6-cyclohexyl-5H-pyrrolo[2,3-b]pyrazin-2-yl)-N,N,3-trimethylbenzenesulfonamide.

13. A pharmaceutical composition, comprising a therapeutically effective amount of a compound according to claim 1 and a pharmaceutically acceptable carrier.

14. A method for treating arthritis, comprising the step of administering a therapeutically effective amount of a compound according to claim 1 to a subject in need thereof.

19. A method for treating a respiratory disorder selected from chronic obstructive pulmonary disorder (COPD), asthma, and bronchospasm, comprising the step of administering a therapeutically effective amount of a compound according to claim 1 to a subject in need thereof
